(19) United States
(12) Patent Application Publication
(10) Pub. No.: US 2026/0232341 A1
(43) Pub. Date: Aug. 13, 2026

(54) HEART VALVE TISSUE MULTI-BLADE CUTTING AND SCORING DEVICE

(71) Applicant: Pi-Cardia Ltd., Rehovot (IL)

(72) Inventor: Benzion Spector, Be’er Yaakov (IL)

(73) Assignee: Pi-Cardia Ltd., Rehovot (IL)

(21) Appl. No.: 19/052,704

(22) Filed: Feb. 13, 2025

(51) Int. Cl. A61B 17/3201; A61B 17/00

(52) U.S. Cl. CPC A61B 17/3201; A61B 17/00234; A61B 2017/00243

(57) ABSTRACT

A heart valve tissue cutting device includes a housing formed with an elongate opening, and first and second splitting elements pivotally coupled to the housing, and arranged to be in a stowed position, in which the first and second splitting elements are collapsed towards the housing, and in a deployed position, in which the first and second splitting elements are deployed outwards from the housing. The first splitting element includes a cutting edge that terminates in a sharp tip which faces towards the housing. The second splitting element includes a cutting edge which faces away from the housing, and the cutting edges of the first and second splitting elements meet at a junction.

### FIELD OF THE INVENTION

The present invention generally relates to devices and methods for transcatheter modification of body tissue such as heart valve leaflets tissue, e.g., aortic, mitral, tricuspid valve leaflets.

### BACKGROUND OF THE INVENTION

PCT Patent Application WO 2020/234763 describes a transcatheter valve laceration device and method, which can be used to perform BASILICA (Bioprosthetic or native Aortic Scallop Intentional Laceration to prevent Iatrogenic Coronary Artery obstruction), or a LAMPOON procedure (for mitral). The device is a cutting or splitting device with attention to preventing damage to neighboring tissues. The device can be implemented in other cardiologic procedures, such as tricuspidization of a bicuspid valve (turning a bicuspid valve into a tricuspid valve by cutting or splitting one of the bicuspid leaflets into two leaflets) or tricuspidization of a quadricuspid valve (lacerating one of the leaflets to turn the valve into a tricuspid valve) or splitting AML (anterior mitral leaflet) to prevent LVOTO (left ventricle outflow tract obstruction), thereby preparing the patient for safe transcatheter aortic or mitral valve replacement (TAVR/TMVR), or for other procedures that involve modifying cardiac/blood vessel tissue.

### SUMMARY

The present invention seeks to provide another device and method for cutting heart valve tissue, such as but not limited to, aortic valve tissue, as is described more in detail hereinbelow. The invention includes more than one cutting blade, wherein one of the blades may be used to initially puncture or stab tissue, and then the tissue may be sliced at the junction of two cutting blades, which can also cut similarly to the action of a scissor. The device includes a scoring tool and an anvil, which can be used to score calcifications, such as on a leaflet positioned between the scoring tool and the anvil.

The device of the invention is particular useful for lacerating the aortic valve leaflets by delivery via the aorta; however, the invention can be used to cut any heart tissue from various approaches and for cutting heart tissue at other valves or portions of the heart.

The terms “cutting” and “scoring” refer to any kind of reduction in size or any modification in shape or form, such as but not limited to, cutting, scoring, splitting, lacerating, slicing, fracturing, chopping and the like, and the terms are used interchangeably throughout.

There is thus provided in accordance with a non-limiting embodiment of the invention a heart valve tissue cutting device including a housing formed with an elongate opening, and first and second splitting elements pivotally coupled to the housing, and arranged to be in a stowed position, in which the first and second splitting elements are collapsed towards the housing, and in a deployed position, in which the first and second splitting elements are deployed outwards from the housing, wherein the first splitting element includes a cutting edge that terminates in a sharp tip which faces towards the housing, and the second splitting element includes a cutting edge which faces away from the housing, and the cutting edges of the first and second splitting elements meet at a junction.

In accordance with a non-limiting embodiment of the invention the first and second splitting elements are pivotally coupled to each other.

In accordance with a non-limiting embodiment of the invention the first splitting element is coupled to a splitter support arm.

In accordance with a non-limiting embodiment of the invention a scoring anvil is pivotally coupled to the housing, and arranged to be in a stowed position, in which the scoring anvil is collapsed towards the housing, and in a deployed position, in which the scoring anvil is deployed outwards from the housing, and wherein the first splitting element is arranged for axial movement towards the scoring arm.

In accordance with a non-limiting embodiment of the invention a positioning arm is pivotally coupled to the housing, and arranged to be in a stowed position, in which the positioning arm is collapsed towards the housing, and in a deployed position, in which the positioning arm is deployed outwards from the housing.

In accordance with a non-limiting embodiment of the invention a protective bumper tip is coupled to a non-hinged end of the positioning arm.

In accordance with a non-limiting embodiment of the invention the scoring anvil is pivotally coupled to the positioning arm.

There is provided in accordance with a non-limiting embodiment of the invention a method for cutting a heart valve tissue by using the first splitting element to initially puncture or stab the heart valve tissue, and then axially moving the first and second splitting elements to slice the heart valve tissue at the junction of the first and second splitting elements. In accordance with a non-limiting embodiment of the invention the method further includes positioning a calcification between the first splitting element and the scoring anvil, and then moving the first splitting element against the calcification to score the calcification.

### BRIEF DESCRIPTION OF DRAWINGS

The present invention will be understood and appreciated more fully from the following detailed description, taken in conjunction with the drawings in which:

FIG. 1 is a simplified side view illustration of a heart valve tissue cutting device, in accordance with a non-limiting embodiment of the present invention.

FIGS. 2A, 2B, 2C and 2D are simplified side view illustrations of the heart valve tissue cutting device, respectively in a closed (stowed or sheathed) position (FIG. 2A), with a positioning arm deployed radially outwards and with a scoring anvil deployed under the positioning arm (FIG. 2B), first (upper) and second (lower) splitting elements deployed radially outwards (FIG. 2C), and the first and second splitting elements having been moved axially so the first splitting element can abut against the scoring anvil (FIG. 2D).

FIGS. 3A and 3B are simplified left and right side view illustrations, respectively, of the first and second splitting elements in the deployed position.

FIG. 3C is a simplified right side view illustration of the first and second splitting elements in the stowed position.

FIGS. 4A and 4B are simplified left side view illustrations of the first and second splitting elements in the deployed and stowed positions, respectively, showing guiding structure for respectively deploying and collapsing the splitter support arm of the splitting elements.

FIG. 4C is a simplified perspective illustration of a multiple-hinge support structure for deploying and collapsing the scoring anvil.

FIGS. 5A, 5B, and 5C are simplified perspective illustrations of the positioning arm, showing that it may be constructed of two concentric tubular elements which increase the stiffness of the positioning arm. The drawings also show a protective bumper tip and show the tissue gripping structure of the positioning arm, such as a saw tooth edge, for improved gripping of the leaflet tissue during cutting or scoring of the tissue.

FIGS. 6A, 6B, 6C, 6D and 6E are simplified pictorial illustrations of the heart valve tissue cutting device, delivered to the heart and being used to cut heart valve tissue.

### DETAILED DESCRIPTION

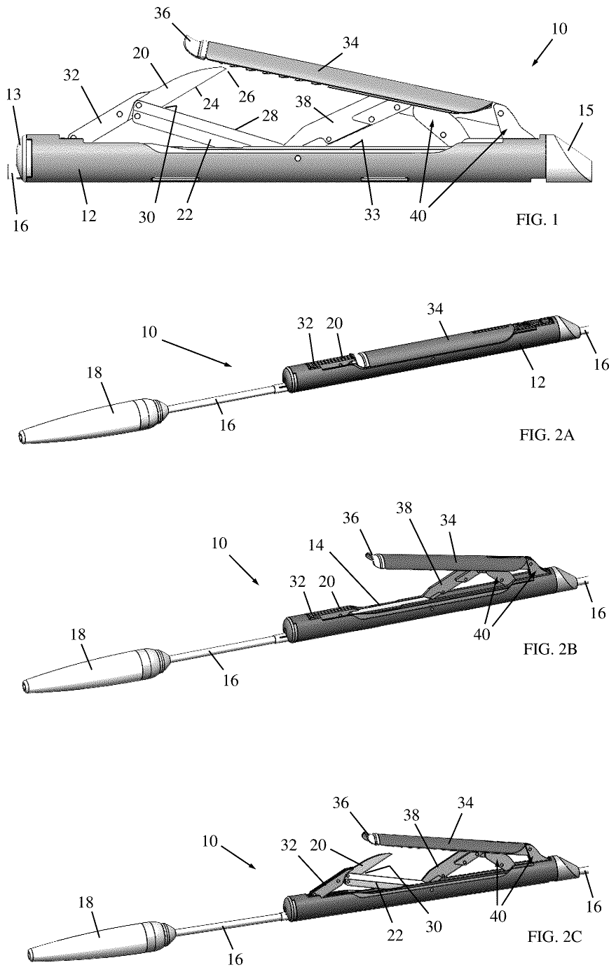

Reference is now made to FIG. 1, which illustrates a heart valve tissue cutting device 10, in accordance with a non-limiting embodiment of the present invention.

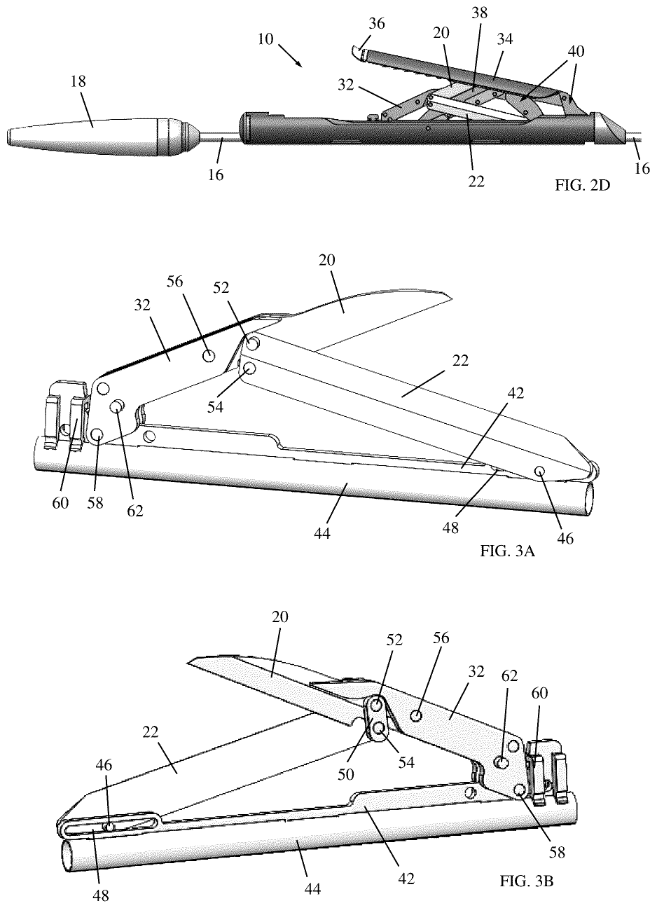

The heart valve tissue cutting device 10 is particularly useful for cutting aortic valve tissue (such as aortic valve leaflets) and can be delivered via the aorta to cut the aortic valve tissue by initially stabbing or puncturing the tissue with a stabbing or puncturing element of a cutting blade, as described below, and then axially slicing the tissue by moving the cutting blade, such as in the direction back to the aorta. For such an approach, the distal end is the left end in FIG. 1 and FIGS. 2A-2D, and the proximal end is the right end in FIG. 1 and FIGS. 2A-2D. It should be understood that distal and proximal are relative, non-limiting terms, and the invention can be used in other orientations. The invention is not limited to the aortic valve leaflets. The device of the invention can be implemented in other cardiologic procedures, such as cutting or slicing mitral valve leaflets, tricuspidization of a bicuspid valve (turning a bicuspid valve into a tricuspid valve by cutting or splitting one of the bicuspid leaflets into two leaflets) or tricuspidization of a quadricuspid valve (lacerating one of the leaflets to turn the valve into a tricuspid valve) or splitting AML (anterior mitral leaflet) to prevent LVOTO (left ventricle outflow tract obstruction), thereby preparing the patient for safe transcatheter aortic or mitral valve replacement (TAVR/TMVR), or for other procedures that involve modifying cardiac/blood vessel tissue.

The heart valve tissue cutting device 10 may include a (e.g., slender, cylindrical) housing 12 formed with an elongate opening 14 (shown in FIG. 2B), which may have rounded ends. The housing 12 may be made of medical grade stainless steel or any other suitable material. Device 10 may be delivered over a guidewire that passes through a guidewire lumen 16, as is known in the art.

As seen in FIGS. 2A-2D, an insertion assist member 18 may be coupled to an end (e.g., distal end) of guidewire lumen 16. The insertion assist member 18 may have a tapered shape that narrows at the distal end. This may help in inserting the device through tissue or body lumens.

The housing 12 may include first and second end caps 13 and 15, as seen in FIG. 1. The first end cap 13 (which is distal in the non-limiting sense of the drawings) may be rounded and the second end cap 15 may have a tilted portion that is tilted towards the direction of first end cap 13. The second end cap 15 may serve as an external sheath guide.

The heart valve tissue cutting device 10 may include first (upper in the non-limiting sense of the drawings) and second (lower in the non-limiting sense of the drawings) splitting elements 20 and 22, respectively. The first splitting element 20 may include a cutting edge 24 that terminates in a sharp tip 26 which faces towards housing 12. The second splitting element 22 may include a cutting edge 28 which faces away from housing 12. The first and second splitting elements 20 and 22 may be pivotally coupled to each other, and the first splitting element 20 may be coupled to a splitter support arm 32. It is seen that the cutting edges of first and second splitting elements 20 and 22 meet at a junction 30. As will be explained below, first splitting element 20 may be used to initially puncture or stab tissue, and then the tissue may be sliced at the junction 30 of two first and second splitting elements 20 and 22, which can also cut similarly to the action of a scissor.

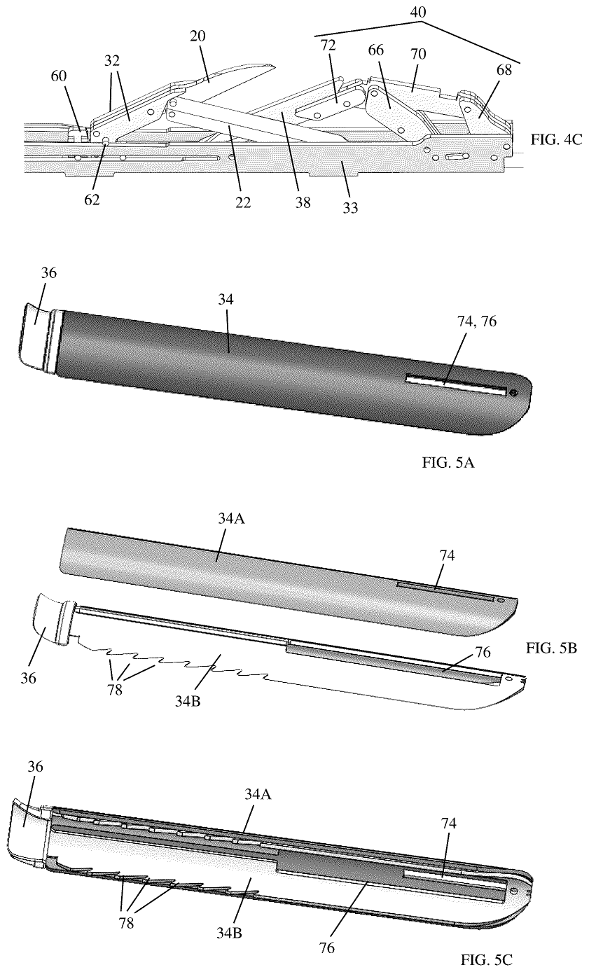

The heart valve tissue cutting device 10 may include a positioning arm 34 pivoted to housing 12 by a multiple-hinge mechanism 40 (as described below with reference to FIG. 4C). A protective bumper tip 36 may be coupled to the non-hinged end of positioning arm 34. The bumper tip 36 may be made of a soft elastomeric material, or any other suitable material, and may be helpful to protect neighboring tissue during the surgical procedure. Without limitation, bumper tip 36 may flare outwards like a partial trumpet shape.

The heart valve tissue cutting device 10 may include a scoring anvil 38. As will be described below, first splitting element 20 can abut against the scoring anvil 38 for scoring calcifications on heart tissue such as leaflets. The scoring anvil 38 may be pivoted to housing 12 by means of the multiple-hinge mechanism 40.

The first and second splitting elements 20 and 22, and the scoring anvil 38 and multiple-hinge mechanism 40 may all be arranged to be deployed by a linkage mechanism along a guide rail 33 (seen better later in FIGS. 4A-4C).

Reference is now made to FIGS. 2A, 2B, 2C and 2D, which illustrate progressive stages in deployment of the heart valve tissue cutting device 10.

FIG. 2A illustrates the device 10 in a closed (stowed or sheathed) position. FIG. 2B illustrates positioning arm 34 deployed radially outwards and scoring anvil 38 deployed under the positioning arm 34. FIG. 2C illustrates first and second splitting elements 20 and 22 deployed radially outwards. FIG. 2D illustrates the first and second splitting elements 20 and 22 having been moved axially (to the right in the sense of the drawing) so the first splitting element 20 abuts against the scoring anvil 38.

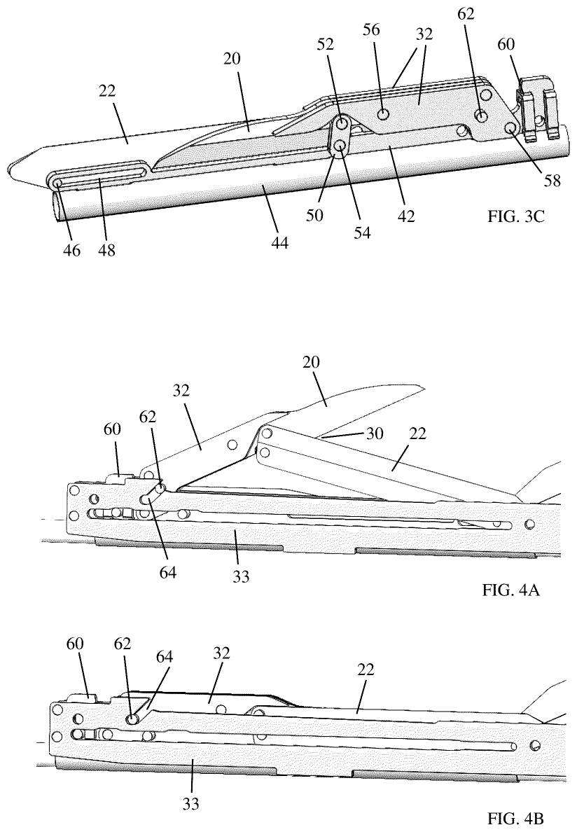

Reference is now made to FIGS. 3A and 3B, which illustrate the first and second splitting elements 20 and 22 in the deployed position, and to FIG. 3C, which illustrates them in the stowed position. It is seen that a central rail 42 may be welded or otherwise coupled to a tube 44. The central rail 42 and the tube 44 serve as a base activating structure for the splitting elements 20 and 22, This base activating structure may be coupled to the guidewire lumen 16 of FIG. 1, such as by means of two rings (not shown). The guidewire lumen 16 (not shown here) may thus serve as a lumen for actuating wires and the like that are coupled to the base activating structure for deploying and contracting the first and second splitting elements 20 and 22.

Second splitting element 22 may be pivotally coupled to central rail 42 by means of a pin 46 that slides in a channel 48. Second splitting element 22 may be pivotally coupled to first splitting element 20 by means of a link element 50 which is coupled by a first pin 52 to first splitting element 20. A second pin 54 may be coupled to second splitting element 22. The purpose of second pin 54 is to help close the first and second splitting elements 20 and 22 to the contracted position and keep them parallel to each other in the closed position, and also prevent any shaking of the first and second splitting elements 20 and 22.

The first splitting element 20 may be coupled to splitter support arm 32 by a rivet 56. As seen best in FIGS. 3B and 3C, splitter support arm 32 includes a pair of arms and first splitting element 20 is sandwiched between these two arms. However, the invention is not limited to this construction, and a single splitter support arm 32 coupled to first splitting element 20 may be used.

The splitter support arm 32 may be pivotally coupled to central rail 42 by means of a pin 58. The splitter support arm 32 may be arranged to abut against a stop 60 secured to central rail 42. Stop 60 defines the limit the splitter support arm 32 can be deployed outwards.

Reference is now made to FIGS. 4A and 4B, which illustrate the first and second splitting elements 20 and 22 in the deployed and stowed positions, respectively, and which illustrate a guiding structure for respectively deploying and collapsing the splitter support arm 32 of the splitting elements 20 and 22.

Without limitation, the guiding structure may include a guide pin 62 secured to splitter support arm 32 that is received in a groove 64 formed in a portion of guide rail 33. In the collapsed position of FIG. 4B, guide pin 62 is fully seated in a rounded end of groove 64. In the deployed position of FIG. 4A, guide pin 62 is at the open end of groove 64.

Reference is now made to FIG. 4C, which illustrates the multiple-hinge support structure or mechanism 40 for deploying and collapsing the scoring anvil 38, in accordance with a non-limiting embodiment of the invention.

The multiple-hinge support structure or mechanism 40 may include a first hinge member 66 and a second hinge member 68, axially separated from each other and both of which have an end pivotally coupled to guide rail 42. The first hinge member 66 and second hinge member 68 may be both pivotally coupled to opposite ends of a third hinge member 70. The third hinge member 70 may be pivotally coupled to a link member 72, which is secured to scoring anvil 38.

Reference is now made to FIGS. 5A, 5B, and 5C, which illustrate the positioning arm 34, in accordance with a non-limiting embodiment of the invention. Positioning arm 34 may be constructed of first and second concentric semi-tubular elements 34A and 34B, which may help increase the stiffness of the positioning arm 34. In the non-limiting sense of the drawing, they are the upper and lower semi-tubular elements 34A and 34B. In this construction, a first guide slot 74 may be formed in first (upper) semi-tubular element 34A, and a second guide slot 76, aligned with slot 74, may be formed in second (lower) semi-tubular element 34B. The guide slots 74 and 76 may provide guidance for the first splitting element, as is now explained. The structure of the first and second splitting elements 20 and 22 (not shown in FIGS. 5A-5C) is not infinitely stiff; rather there is some degree of freedom of movement. The first splitting element (the one that punctures the tissue) may deflect or otherwise move slightly sideways while puncturing and splitting the tissue, due to the resistance of the tissue. Any deflection is prevented by the sharp tip of the first splitting element entering the thin guide slot 76 of the inner semi-tubular element 34B. The guide slot 76 and guide slot 74 guide the movement of the blade of the first splitting element while splitting. This guide structure also causes partial closure of the scissors structure of the first and second splitting elements, which reduces the angle between the first and second splitting elements 20 and 22 to improve the cutting action at the junction 30 (FIG. 1).

Positioning arm 34 may include tissue gripping structure 78, such as a saw tooth edge, for improved gripping of the leaflet tissue during cutting (e.g., splitting) of the tissue. The tissue gripping structure 78 may be formed along the lower edge of either one or both of first and second concentric semi-tubular elements 34A and 34B.

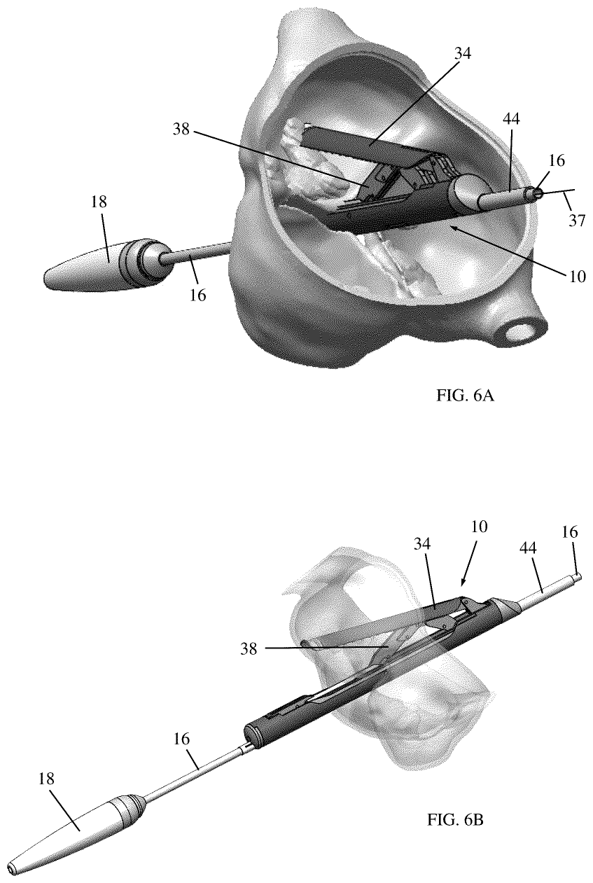

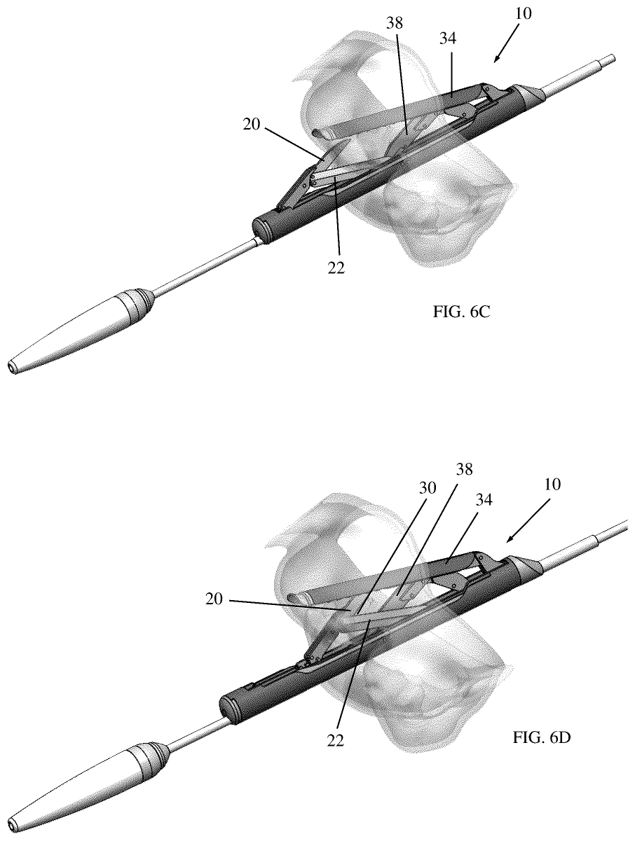

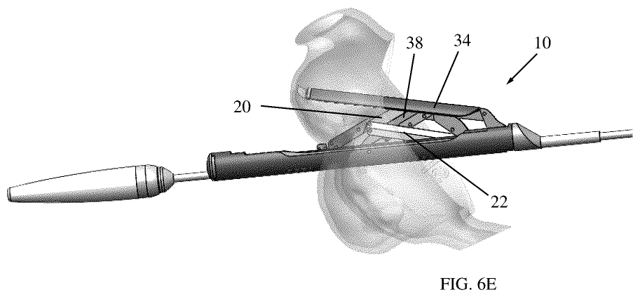

Reference is now made to FIGS. 6A-6E, which illustrate use of the heart valve tissue cutting device 10 for cutting aortic valve tissue (such as aortic valve leaflets).

In FIGS. 6A and 6B, device 10 has been delivered over a guidewire 37 (shown in FIG. 6A) that passes through guidewire lumen 16 via the aorta to the aortic valve. Positioning arm 34 is then deployed outwards along with the scoring anvil 38 (corresponding to FIG. 2B). The deployment may be done by actuator wires coupled to the device and manipulated by a manipulating handle held by the surgeon. Alternatively, the positioning arm 34 may be spring-loaded and automatically deploy upon unsheathing, and the scoring anvil 38 deploys together with the positioning arm 34.

In FIG. 6C, the first and second splitting elements 20 and 22 have been deployed radially outwards (corresponding to FIG. 2C). The

In FIG. 6D, the first and second splitting elements 20 and 22 have been moved axially towards scoring anvil 38. The first splitting element 20 initially punctures or stabs the tissue, and then the tissue may be sliced at the junction 30 of the first and second splitting elements 20 and 22 by the axial movement of the first and second splitting elements 20 and 22.

In FIG. 6E, the first and second splitting elements 20 and 22 have been moved further axially so the first splitting element 20 abuts against the scoring anvil 38 (corresponding to FIG. 2D). In this manner, the first splitting element 20 can score calcifications found on leaflets or other tissue.

The scoring of the calcification helps in splitting the leaflet by creating a smooth split line through the calcified leaflet in preparation for splitting the leaflet. The split line that is scored into the calcified leaflet defines the cut line for the splitting elements and safely reduces the amount of emboli created during breaking of calcium on the leaflet.

1. A heart valve tissue cutting device comprising:
a housing formed with an elongate opening; and
first and second splitting elements pivotally coupled to said housing, and arranged to be in a stowed position, in which said first and second splitting elements are collapsed towards said housing, and in a deployed position, in which said first and second splitting elements are deployed outwards from said housing;
wherein said first splitting element comprises a cutting edge that terminates in a sharp tip which faces towards said housing, and said second splitting element comprises a cutting edge which faces away from said housing, and said cutting edges of said first and second splitting elements meet at a junction.

2. The heart valve tissue cutting device according to claim 1, wherein said first and second splitting elements are pivotally coupled to each other.

3. The heart valve tissue cutting device according to claim 1, wherein said first splitting element is coupled to a splitter support arm.

4. The heart valve tissue cutting device according to claim 1, further comprising a scoring anvil pivotally coupled to said housing, and arranged to be in a stowed position, in which said scoring anvil is collapsed towards said housing, and in a deployed position, in which said scoring anvil is deployed outwards from said housing, and wherein said first splitting element is arranged for axial movement towards said scoring arm.

5. The heart valve tissue cutting device according to claim 1, further comprising a positioning arm pivotally coupled to said housing, and arranged to be in a stowed position, in which said positioning arm is collapsed towards said housing, and in a deployed position, in which said positioning arm is deployed outwards from said housing.

6. The heart valve tissue cutting device according to claim 5, wherein a protective bumper tip is coupled to a non-hinged end of said positioning arm.

7. The heart valve tissue cutting device according to claim 4, further comprising a positioning arm pivotally coupled to said housing, and arranged to be in a stowed position, in which said positioning arm is collapsed towards said housing, and in a deployed position, in which said positioning arm is deployed outwards from said housing, and wherein said scoring anvil is pivotally coupled to said positioning arm.

8. A method for cutting a heart valve tissue comprising using the device of claim 1 by using said first splitting element to initially puncture or stab the heart valve tissue, and then axially moving said first and second splitting elements to slice the heart valve tissue at said junction of said first and second splitting elements.

9. The method according to claim 8, further comprising a scoring anvil pivotally coupled to said housing, and arranged to be in a stowed position, in which said scoring anvil is collapsed towards said housing, and in a deployed position, in which said scoring anvil is deployed outwards from said housing, and wherein said first splitting element is arranged for axial movement towards said scoring arm; and further comprising using said scoring anvil to position a calcification between said first splitting element and said scoring anvil, and moving said first splitting element against the calcification to score the calcification and define a splitting line as preparation for splitting the heart valve tissue.
